(19) United States
(12) Patent Application Publication
(10) Pub. No.: US 2011/0130687 A1
(43) Pub. Date: Jun. 2, 2011

(54) Safety detection method for ultrasonic treatment device

(76) Inventor: Hsin-Yi Lin, Shindian City (TW)

(21) Appl. No.: 12/591,759

(22) Filed: Dec. 1, 2009

(51) Int. Cl. A61N 7/00

(52) U.S. Cl. 601/2

(57) ABSTRACT

A detection method for an ultrasonic treatment device is disclosed. A current value presently flowing through an output circuit of the ultrasonic treatment device is detected when a probe of the ultrasonic treatment device is in contact with a surface of a user's skin. Whether or not the change in the current values maintains within a predetermined current error range is determined, and whether or not a sampling holding time during which the change in the current values maintains within the current error range exceeds a predetermined time reference value is also determined. When the probe is determined to be under abnormal conditions according to the current error range, time reference value and the upper limit value of working time, the probe is stopped from generating ultrasonic energy, thereby lowering the risks for the user and preventing the user from injuries.

### FIELD OF THE INVENTION

This invention relates to the design for safely using an ultrasonic treatment device, and particularly to a detection method for an ultrasonic treatment device.

### BACKGROUND OF THE INVENTION

Conventional heat therapy for treatments of bone, muscle and other sports injuries is nothing more than covering injuries with a hot towel, steaming, or directly soaking the injured parts in hot water. However, these methods can only conduct the heat to the surface of the skin, thus is unable to conduct the heat deeply into the muscle and bone, particularly to treat maladies of the joints and deep seated muscles.

Therefore, the industry has developed an ultrasonic treatment device that uses the resonance principles of ultrasonic waves to transfer heat into the bones and muscles. The ultrasonic wave is transferred to the injured bones, muscles or joints to enhance the metabolism of the bodies that can reduce and alleviate the pains and cure the parts. Moreover, the ultrasonic wave can promote the permeation of the ointment into the muscles and bones that is helpful to treatment.

Ultrasonic treatment devices in the prior art mainly comprise a probe that contacts the surface of the skin and generates ultrasonic energy. Ultrasonic treatment devices are effective in medical applications, but in contrast, may also cause risks and injuries, thus a physician's approval is generally needed for this form of treatment to be used. If the user is not a specialist, they are unable to safely and effectively use the ultrasonic treatment device.

### SUMMARY OF THE INVENTION

In accordance with the foregoing, there are many dangers and risks when using an ultrasonic treatment device. The biggest problem is being unable to use the device at a fixed point for a long period of time. The device must be continuously maintained in a state of motion or sliding during use. If left in contact in the same position for a long period of time, the wave energy accumulated would cause subcutaneous tissue or organs to be injured. Furthermore, when the energy of the ultrasonic treatment device is under a no-load condition (zero load), the energy in the probe is unable to dissipate, resulting in the heating up of the probe which could very easily cause burns when a user is not paying attention.

Therefore, an object of the present invention is to provide an detection method for an ultrasonic treatment device to prevent the'user from injuries or risks during use of the device, enabling the user to safely and effectively use the ultrasonic treatment device.

The solution adopted in the present invention is to provide a detection method for an ultrasonic treatment device. A current value presently flowing through an output circuit of the ultrasonic treatment device is detected when a probe of the ultrasonic treatment device is in contact with a surface of a user's skin. Whether or not a change in the current values maintains within a predetermined current error range is determined, and whether or not a sampling holding time during which the change in the current values maintains within the current error range exceeds a predetermined time reference value is also determined. When the probe is determined to be under abnormal conditions according to the current error range, time reference value and the upper limit value of working, the probe is stopped from generating ultrasonic energy, thereby lowering the risks for the user and preventing the user from injuries.

In a preferred embodiment, whether or not the change in the current values maintains within the current error range Rt is determined by first comparing the detected current value with a current value detected from the last sampling in order to obtain a current change. Then, it is determined whether or not the current change is smaller than the current error range and whether or not the ultrasonic energy should be stopped.

The techniques employed by the present invention can determine whether or not the probe of the ultrasonic treatment device is in motion or sliding according to set parameters during use, whether or not it is staying at a point unmoving for a long period of time, or if it is not in contact with the surface of the skin causing a zero load to happen. The moment it is determined that any of the above is at risk to the user or will cause injuries, the ultrasonic energy is stopped or paused from being emitted to prevent the user from injuries or danger during use of the device. Therefore, the user is able to safely and effectively use the ultrasonic treatment device.

### BRIEF DESCRIPTION OF THE DRAWINGS

FIG. 1 is a schematic diagram illustrating a control circuit of an ultrasonic treatment device.

FIG. 2 illustrates a control flowchart according to a first embodiment of the present invention.

FIG. 3 illustrates a control flowchart according to a second embodiment of the present invention.

### DETAILED DESCRIPTION OF THE PREFERRED EMBODIMENTS

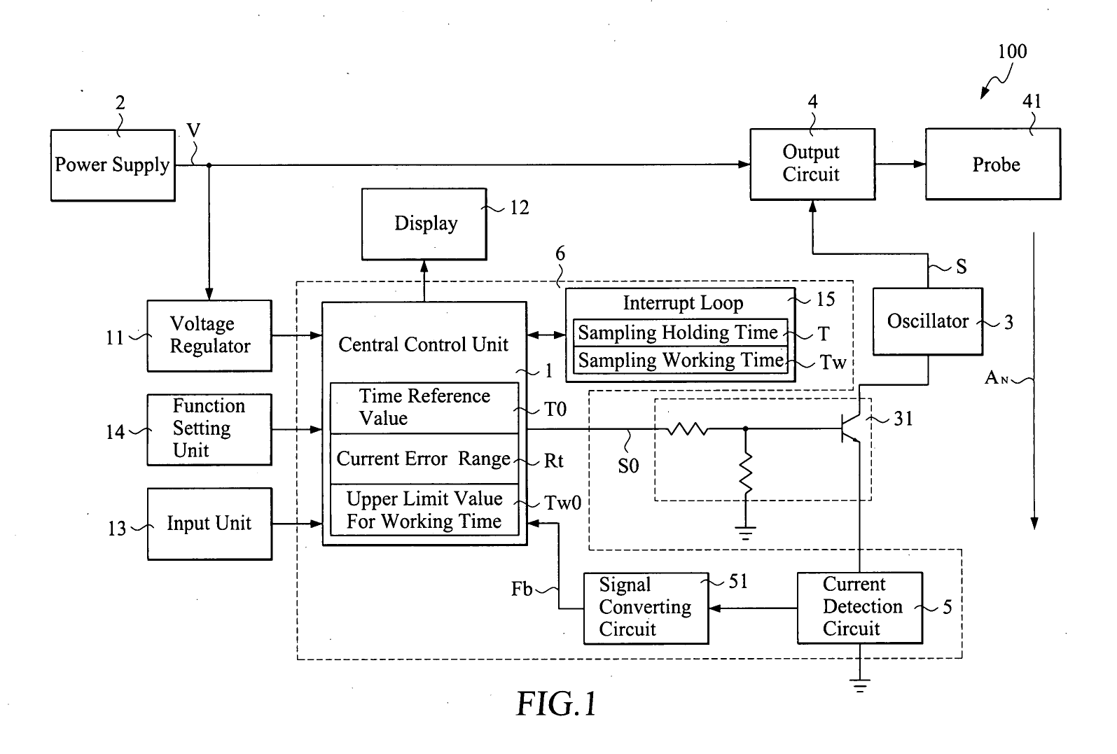

FIG. 1 illustrates a control circuit of an ultrasonic treatment device 100. The ultrasonic treatment device 100 comprises a central control unit 1, a power supply 2, an oscillator 3, an output circuit 4, and a current detection circuit 5.

The central control unit 1 controls the entire ultrasonic treatment device and is connected to a voltage regulator 11, a display 12, an input unit 13, a function setting unit 14, and an interrupt loop 15. The voltage regulator H is connected to the power supply 2 to regulate an input power source V provided by the power supply 2, and then supply the input power source V to the central control unit 1. The display 12 provides the function for displaying images, graphics, data, information, etc. The input unit 13 provides input commands. The function setting unit 14 enables the setting of high/low mode, the timer, and the memory for emitting the ultrasonic energy. The interrupt loop 15 provides various interrupt timing.

The oscillator 3 is connected to the central control unit 1 via the oscillator switch loop 31 and generates a sequence of oscillator signals S to the output circuit 4. The output circuit 4 receives the oscillator signals S and generates ultrasonic energy through a probe 41.

The current detection circuit 5 is connected to the output circuit 4 via the oscillator switch loop 31 and the oscillator 3, and is also connected to the central control unit 1 via the signal converting circuit 51. The current detection circuit 5 is used to detect a current value A_{N} flowing through the output circuit 4, which is converted by the signal converting circuit 51 into a current signal Fb and then sent to the central control unit 1. At this point, the central control unit 1, the interrupt loop 15, the current detection circuit 5, and the signal converting circuit 51 forms an intelligent detection loop 6 to carry out safety detection during use.

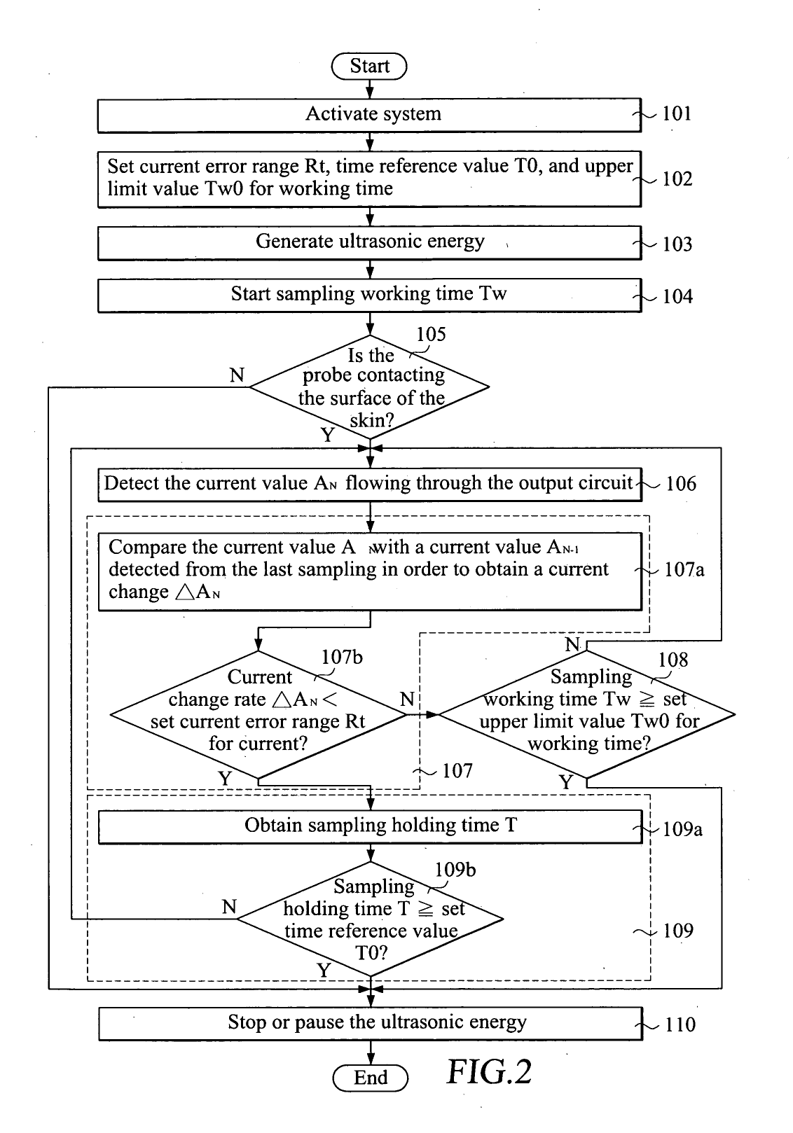

Referring to FIG. 2, which illustrates a control flowchart according to a first embodiment of the present invention. Please also refer to FIG. 1. The ultrasonic treatment device is first activated by supplying an input power source V from the power supply 2 (Step 101). Before starting ultrasonic therapy, firstly the necessary parameters are set to the ultrasonic treatment device, wherein the parameters comprise a current error range Rt, a time reference value T0, and an upper limit value for working time Tw0 (Step 102). These parameters can be set by the input unit 13, and can also be pre-stored into a memory. After completing the settings, the oscillator 3 is activated, which generates the oscillator signals S to the output circuit 4 and then to the probe 41 to generate the ultrasonic energy (Step 103). At this point, the interrupt loop 15 simultaneously starts timing and obtaining the sampling working time Tw during which the ultrasonic energy is generated (Step 104).

After the ultrasonic energy is generated from the probe 41, detection of a load is first carried out to determine whether or not the probe 41- is in contact with a surface of a skin (Step 105). During practical application, from the changes in the current values A_{N} flowing through the output circuit 4 detected by the current detection circuit 5, it is able to determine whether or not the probe 41 is in contact with the surface of the skin. The detected current value A_{N} when the probe 41 is in contact with the surface of the skin is different from that when the probe 41 is not in contact with the surface. By applying this principle, it can determine whether or not the probe 41 is in contact with the surface of the skin. Additionally, when the probe 41 is under a no-load condition (not in contact with the surface of the skin), the wave energy is unable to dissipate and causes a high temperature. Therefore, by detecting a temperature of the probe 41, it is able to determine whether or not the probe 41 is in contact with the surface of the skin.

When it is determined that the probe 41 is not in contact with the surface of the skin, the probe 41 is stopped or paused from generating ultrasonic energy. This is achieved by the central control unit 1 which generates a control signal SO to the oscillator switch loop 31 and causes the oscillator switch loop 31 to turn to an open state which disables the oscillator 3, thereby stopping or pausing the probe 41 from generating ultrasonic energy. Of course, the input power source V provided by the power supply 2 can also be directly cut off to stop the generation of ultrasonic energy.

When it is determined that the probe 41 is in contact with the surface of the skin, a motion detection is performed. The method for motion detection comprises firstly, using the current detection circuit 5 to detect the current value A_{N} presently flowing through the output circuit 4 (Step 106), and then determining whether or not the changes in the detected current value A_{N} are maintained within the current error range Rt (Step 107).

When the ultrasonic treatment device 100 performs oscillations and generates ultrasonic energy, the changes in loads causes the current values A_{N} to change significantly. According to this principle, the current error range Rt preset within the ultrasonic treatment device becomes the reference value. If the change in the detected current values A_{N} exceed the current error range Rt, the range of change in the current value A_{N} is wide enough, thereby indicating that the probe 41 is maintained in a state of motion or sliding. If the change in the detected current values A_{N} stays within the current error range Rt, this indicates that the probe 41 is not moving or sliding and is staying in the same position for a long period of time.

In the present embodiment, whether or not the change in the current values A_{N} are maintained within the current error range Rt is determined by first comparing the detected current value A_{N} with a current value A_{N-1} detected from the last sampling in order to obtain a current change ΔA_{N} (Step 107a). Then, it is determined whether or not the current change ΔA_{N} is smaller than the current error range Rt (Step 107b). Comparing the results of the present sample and the last sample allows the exclusion of some errors caused by variables affected by time, thereby enabling more accurate determination. Moreover, the current error range Rt is set in the central control unit 1 which determines the changes of the current values A_{N}. Besides, by means of the circuit design, the signal converting circuit 51 is also used to compare the changes in the current values A_{N} with the current error range Rt while a signal is converted by the signal converting circuit 51. Then, the current condition signal Fb fed from the signal converting circuit 51 is transmitted to the central control unit 1 to be subsequently determined.

When the changes in the detected current value A_{N} exceed the current error range Rt (staying in a state of motion or sliding), it is determined whether or not the sampling working time Tw during which the ultrasonic energy is generated from the probe 41 obtained by the interrupt loop 15 reaches a predetermined upper limit value for working time Tw0 (Step 108). When the sampling working time Tw during which the ultrasonic energy is generated from the probe 41 reaches the upper limit value for working time Tw0, the probe 41 is stopped from generating ultrasonic energy to prevent the excessive working time that would cause accidents. The probe 41 is stopped by disabling the oscillator 3 by the central control unit 1, and also by directly cutting off the input power source V provided by the power supply 2.

When the detected changes in the current value A_{N} are maintained within the current error range Rt (not in motion or sliding), it is determined whether or not the sampling holding time T during which the change in the current values A_{N} maintained within the current error range Rt exceeds the time reference value T0 (Step 109). This can be achieved by obtaining the sampling holding time T by the interrupt loop 15 (Step 109a), and by determining whether or not the sampling holding time T exceeds the time reference value T0 by the central control unit 1 (Step 109b). When the sampling holding time T is determined to exceed the time reference value T0, the probe 41 is stopped from generating ultrasonic energy (Step 110) to prevent the probe 41 from staying in contact in the same position for a long period of time, letting wave energy build up, and cause injuries. The probe 411 is stopped by disabling the oscillator 3 by the central control unit 1, and also by directly cutting off the input power source V provided by the power supply 2.

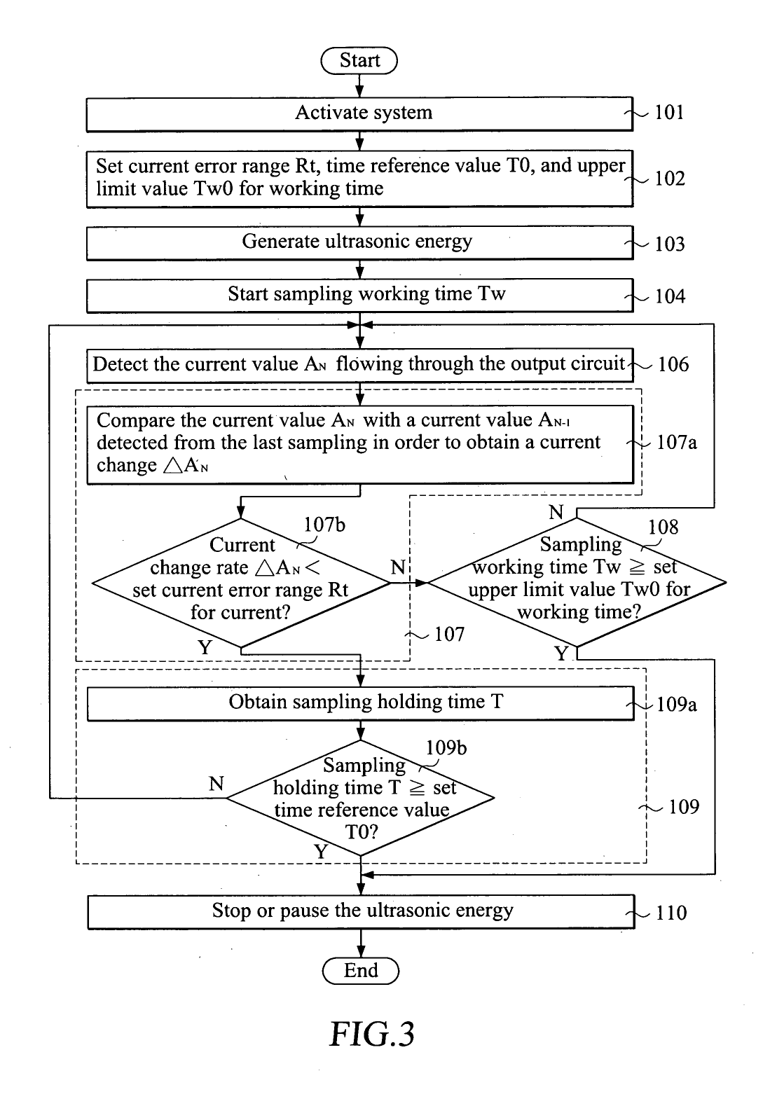

Referring to FIG. 3, which illustrates a control flowchart of according to a second embodiment of the present invention. The steps of this embodiment are essentially the same as that of the aforementioned first embodiment, thus the same steps are designated with the same reference numerals. The difference is that the step for determining whether or not the probe 41 is in contact with the surface of the skin is eliminated, and the subsequent detection of motion or sliding is proceeded.

Although the present invention has been described with reference to the preferred embodiments thereof, as well as the best modes for carrying out the present invention, it is apparent to those skilled in the art that a variety of modifications and changes may be made without departing from the scope of the present invention which is intended to be defined by the appended claims.

What is claimed is:

1. A detection method for an ultrasonic treatment device, wherein the ultrasonic treatment device comprises an oscillator, for generating an oscillator signal and an output circuit for receiving the oscillator signal to correspondingly generate an ultrasonic energy through a probe, the method comprises the following steps:
(a) detecting whether or not the probe is in contact with a surface of a skin;
(b) detecting a current value presently flowing through the output circuit when the probe is determined to be in contact with the surface of the skin;
(c) determining whether or not a change in the detected current values maintains within a predetermined current error range;
(d) determining whether or not a sampling holding time during which the change in the current value maintains within the current error range exceeds a predetermined time reference value when the change in the current values is determined to maintain within the current error range; and
(e) stopping the ultrasonic energy when the sampling holding time is determined to exceed the time reference value.

2. The detection method according to claim 1, further comprising a step to set the current error range before Step (a).

3. The detection method according to claim 1, further comprising a step to set the time reference value before Step (a).

4. The detection method according to claim 1, wherein determining whether or not the probe is in contact with the surface of the skin through detection of the changes in the current value flowing through the output circuit in Step (a).

5. The detection method according to claim 1, further comprising a step to stop the probe from generating the ultrasonic energy when the probe is determined to not be in contact with the surface of the skin after Step (a).

6. The detection method according to claim 1, wherein determining whether or not the detected changes in the current value are maintained within the current error range in Step (c); the method comprising the following steps:
(c1) comparing the detected current value with a current value detected by a last time point sample to obtain a current change; and
(c2) determining whether or not the current change is smaller than the current error range.

7. The detection method according to claim 1, further comprising a step to determine whether or not a sampling working time during which the ultrasonic energy is generated from the probe reaches a predetermined upper limit value of working time when the change in the current values is determined to exceed the current error range after Step (c).

8. The detection method according to claim 7, further comprising a step to stop the probe from generating the ultrasonic energy when the sampling working time during which the ultrasonic energy is generated from the probe is determined to reach the predetermined upper limit value of working time after the step to determine whether or not a sampling working time of the ultrasonic energy generated from the probe reaches a predetermined upper limit value.

9. The detection method according to claim 8, wherein the ultrasonic energy is stopped by disabling the oscillator.

10. The detection method according to claim 1, wherein the ultrasonic energy is stopped by disabling the oscillator in Step (e).

11. A detection method for an ultrasonic treatment device, wherein the ultrasonic treatment device comprises an oscillator, for generating an oscillator signal and an output circuit for receiving the oscillator signal to correspondingly generate an ultrasonic energy through a probe, the method comprises the following steps:
(a) detecting a current value presently flowing through the output circuit when the probe is determined to be in contact with a surface of a skin;
(b) comparing the detected current value with a current value detected by a last time point sample to obtain a current change;
(c) determining whether or not the current change is smaller than the current error range;
(d) determining whether or not a sampling holding time during which the change in the current value maintains within the current error range exceeds a predetermined time reference value when the change in the current values is determined to maintain within the current error range; and
(e) stopping the ultrasonic energy when the sampling holding time is determined to exceed the time reference value.

12. The detection method according to claim 11, further comprising a step to set the current error range before Step (a).

13. The detection method according to claim 11, further comprising a step to set the time reference value before Step (a).

14. The detection method according to claim 11, further comprising a step to determine whether or not a sampling working time during which the ultrasonic energy is generated from the probe reaches a predetermined upper limit value when the change in the current values is determined to exceed the current error range after Step (c)

15. The detection method according to claim 14, further comprising a step to stop the probe from generating the ultrasonic energy when the sampling working time during which the ultrasonic energy is generated from the probe is determined to reach the predetermined upper limit value of working time after the step to determine whether or not a sampling working time of the ultrasonic energy generated from the probe reaches the predetermined upper limit value.

16. The detection method according to claim 15, wherein the ultrasonic energy is stopped by disabling the oscillator.

17. The detection method according to claim 11, wherein the ultrasonic energy is stopped by disabling the oscillator in Step (e).
